(19) United States
(12) Patent Application Publication
(10) Pub. No.: US 2006/0194748 A1
(43) Pub. Date: Aug. 31, 2006

(54) Methods for treating disorders induced by H. pylori infections and pharmaceutical compositions for the same

(75) Inventors: Masaaki Minami, Nagoya (JP); Takafumi Ando, Nagoya (JP); Hidemi Goto, Nagoya (JP); Michio Ohta, Nagoya (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: National University Corporation Nagoya University, Nagoya (JP)

(21) Appl. No.: 11/066,476

(22) Filed: Feb. 28, 2005

(51) Int. Cl. A61K 31/7048; A61K 31/43; A61K 31/198

(52) U.S. Cl. 514/29; 514/192; 514/561

(57) ABSTRACT

The present invention provides a pharmaceutical composition for the treatment of disorders induced, caused, and/or mediated by Helicobacter pylori infection, as well as methods of treating the same by administering the pharmaceutical composition of the present invention.

### BACKGROUND OF THE INVENTION

1. Field of the Invention

The present invention provides a pharmaceutical composition for the treatment of disorders induced, caused, and/or mediated by Helicobacter pylori infection. In the context of the present invention, this composition preferably comprises a therapeutically effective amount of (a) glycine or a pharmaceutically acceptable salt thereof, (b) at least one additional Helicobacter pylori therapeutic agent, and a pharmaceutically acceptable carrier or excipient.

Moreover, the present invention is related to a method for the prophylactic and/or therapeutic treatment of disorders induced, caused and/or mediated by Helicobacter pylori infections comprising administering to a subject in need thereof an effective amount of the pharmaceutical compositions of the present invention.

2. Discussion of the Background

Helicobacter pylori is a microaerophilic, gram-negative spiral bacterium that is recognized as a pathogenic bacterium (9, 26). In humans, H. pylori is associated with peptic ulcer, malignant lymphoma, and gastric cancer (39). Approximately 80% of the populations in many developing countries are estimated to be infected with H. pylori (40), whereas about 30% of the populations in developed countries are estimated to be infected with H. pylori (12). Although the mechanism of the pathogenicity of this bacterium has not been clearly explained, eradication of H. pylori greatly contributes to an eminent improvement in these diseases (7, 34, 39). Therefore, the prevailing belief in the art is that, even if a causal relationship between H. pylori and these diseases does not exist, H. pylori plays a predominant role in the onset and/or progression of these diseases.

In Japan, the present first-line treatment for the eradication of H. pylori is a triple-therapy combination with a proton pump inhibitor, clarithromycin (CLR), and amoxicillin (AMX). This combination achieves clinical cure rates of greater than 80% (5). However, the prevalence of CLR resistance varies with geographical location and is generally estimated to be about 10% in Japan (21). Given that CLR has a stronger antibacterial effect on H. pylori than other agents (24), the presence of resistant microbes may result in eradication failure (1). Although various other agents such as metronidazole or tetracycline are candidates for eradication of CLR-resistant strains (17), their clinical use is limited because of side effects or the presence of strains resistant to those drugs as well (36). Thus, a critical need remains in the art for the identification of new, safer alternative antibacterial agents.

Glycine is the simplest amino acid and has been known to possess some antibacterial properties. As such, glycine has been added to foods to serve in antibacterial capacity due to its low toxicity in animals (11, 13, 33). As an example of the antibacterial potential of glycine, Lactococcus lactis subspecies failed to grow in medium containing more than 2% glycine (15). Furthermore, glycine concentrations of 1.5 to 6% resulted in 70 to 90% reductions in growth of Enterococcus faecalis (10). However, heretofore, the efficacy of glycine in H. pylori has not been elucidated.

The postantibiotic effect (PAE) is a phenomenon by which the inhibition of bacterial growth continues after exposure to an antimicrobial agent (8, 19, 25). It is classically defined as the period of bacterial growth suppression that persists after limited exposure of organisms to antimicrobials. The PAE may remain even after drug levels are no longer detectable. The duration of the PAE is of important clinical interest in establishing dosing schedules, particularly given that longer intervals between intermittent dosage schedules may reduce the toxicities of antibiotics (25). To date, however, the PAE of glycine against H. pylori has not been well studied and little information is available.

In view of the foregoing, a critical need still exists in the art for the identification of new antibacterial drugs for the eradication of diseases related to H. pylori infection. It is within this framework that the present applicants have endeavored.

### SUMMARY OF THE INVENTION

It is an object of the present invention to provide a pharmaceutical composition comprising:
(a) a therapeutically effective amount of glycine or a pharmaceutically acceptable salt thereof;
(b) a therapeutically effective amount of at least one additional Helicobacter pylori therapeutic agent;
and a pharmaceutically acceptable carrier or excipient.

In this object and those following, said additional Helicobacter pylori therapeutic agent is amoxicillin and/or clarithromycin. Still further, the pharmaceutical composition may also contain a therapeutically effective amount of a proton pump inhibitor and/or a therapeutically effective amount of a histamine-H2 receptor blocking compound.

Within this object, when amoxicillin is present, the ratio of glycine to amoxicillin ranges from 1.25:1 to 5000:1.

In another object of the present invention is a method for the prophylactic and/or therapeutic treatment of at least one disorder induced, caused or mediated by Helicobacter pylori infection comprising: administrating to a subject in need thereof a therapeutically effective amount of glycine or a pharmaceutically acceptable salt thereof. In an embodiment of this object, the Helicobacter pylori is clarithromycin-resistant Helicobacter pylori. Still further, the disorder induced, caused or mediated by Helicobacter pylori infection is preferably a peptic ulcer, malignant lymphoma, gastric cancer, chronic urticaria, and thrombocytopenia.

Within the methods of the present invention, the mode of administering is by a mode of administration selected from the group consisting of oral administration, parenteral administration, and intraperioneal administration. Further, in the objects of the present invention, the therapeutically effective amount ranges from 500 mg to 30 g of glycine or a pharmaceutically acceptable salt thereof administered one to three times per day.

In yet another object of the present invention is to provide a method for the prophylactic and/or therapeutic treatment of at least one disorder induced, caused or mediated by Helicobacter pylori infection comprising: administrating to a subject in need thereof a therapeutically effective amount of:
(a) glycine or a pharmaceutically acceptable salt thereof; and
(b) an effective amount of at least one additional Helicobacter pylori therapeutic agent.

Within this object, (a) and (b) may be concurrently administered or sequentially administered. In an embodiment of this object, the Helicobacter pylori is clarithromycin-resistant Helicobacter pylori.

In an embodiment of the present invention, in the aforementioned method said additional Helicobacter pylori therapeutic agent is amoxicillin. When said additional Helicobacter pylori therapeutic agent is amoxicillin the therapeutically effective amount ranges from 500 mg to 30 g of glycine or a pharmaceutically acceptable salt thereof and from 25 mg to 750 mg of amoxicillin administered one to three times per day. Further, said additional Helicobacter pylori therapeutic agent may be clarithromycin, a proton pump inhibitor, and/or a histamine-H2 receptor blocking compound.

Thus, in yet another object of the present invention is to provide a method for the prophylactic and/or therapeutic treatment of at least one disorder induced, caused or mediated by Helicobacter pylori infection comprising administering;

(a) a therapeutically effective amount of glycine or a pharmaceutically acceptable salt thereof;

(b) a therapeutically effective amount of amoxicillin and a therapeutically effective amount of clarithromycin; and

(c) a therapeutically effective amount of a proton pump inhibitor.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows a comparison of the inhibitory effects of glycine against CLR-susceptible and CLR-resistant H. pylori strains. H. pylori strains were treated with the indicated concentrations of test compound for 3 days. The mean value for the log number of CFU per milliliter of all CLR-susceptible and all CLR-resistant H. pylori strains at each concentration was plotted. Glycine was used at concentrations of 0, 0.5, 1, 1.5, 2.0, and 2.5 mg/ml. Representative results from five independent experiments are shown. Symbols: ▪, CLR-susceptible strains; ▴, CLR-resistant strains.

FIG. 2 shows the results of the time-kill curve experiment with glycine and H. pylori 26695. H. pylori was inoculated into BB with glycine and incubated at 37° C. for 2 days. Glycine in BB was added to the cultures at the start of incubation. The mean values for the log number of CFU per milliliter versus time for H. pylori were tested with different concentrations of glycine. At 12-h intervals after drug addition, aliquots of each culture were spread onto BB agar for determination of the number of CFU. Representative results from five independent experiments are shown. Symbols: ▪, glycine at 0 mg/ml; ♦, glycine at 1 mg/ml; ▴, glycine at 2.5 mg/ml; ●, glycine at 5 mg/ml; □, glycine at 10 mg/ml.

FIG. 3 shows the inhibitory effects of various concentrations of glycine and AMX on H. pylori 26695. H. pylori was treated with the indicated concentrations of the test compounds for 3 days. AMX was used at concentrations of 0 to 75 ng/ml. Glycine was used at concentrations of 0, 0.5, 1, and 2.5 mg/ml. The mean value for the log number of CFU per milliliter at each concentration was plotted. Representative results from five independent experiments are shown. Symbols: ▪, glycine at 0 mg/ml with AMX; ♦, glycine at 0.5 mg/ml with AMX; ▴, glycine at 1 mg/ml with AMX; ●, glycine at 2.5 mg/ml with AMX.

FIG. 4 shows time-kill curve experiments with glycine-AMX and H. pylori 26695. H. pylori was inoculated into BB with glycine and incubated at 37° C. for 24 h. Glycine and AMX in BB were added to cultures at the start of incubation. The mean values for the log of the number of CFU per milliliter versus time for H. pylori were tested with different concentrations of glycine and AMX. At 6-h intervals after drug addition, aliquots from each culture were spread onto BB agar plates for determination of the numbers of CFU. Representative results from five independent experiments are shown. Symbols: ▪, glycine at 0 mg/ml; ♦, glycine at 10 mg/ml; ▴, at glycine at 2.5 mg/ml with AMX at 7.5 ng/ml; ●AMX at 75 ng/ml.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Glycine is the simplest amino acid and is used as a metabolic product in some bacteria. However, an excess of glycine inhibits the growth of many bacteria, and it is used as a nonspecific antiseptic agent due to its low level of toxicity in animals (11, 13, 33). The effect of glycine on Helicobacter pylori is not precisely known. The present invention was completed in part based on the present inventors' investigation into (i) the effect of glycine on clarithromycin (CLR)-resistant and -susceptible strains of H. pylori, (ii) the effect of glycine in combination with amoxicillin (AMX), and (iii) the postantibiotic effect (PAE). As shown in the Examples, the MIC at which 90% of strains are inhibited for glycine was almost 2.5 mg/ml for 31 strains of H. pylori, including CLR-resistant strains. Further, the combination of AMX and glycine showed synergistic activity, with the minimum bactericidal concentration of AMX with glycine decreasing to 1/10 that of AMX alone. Glycine showed no PAE against H. pylori. These results demonstrate the utility of glycine as an antimicrobial agent against H. pylori independently or in combination with antibacterial drugs for the treatment of H. pylori-associated diseases.

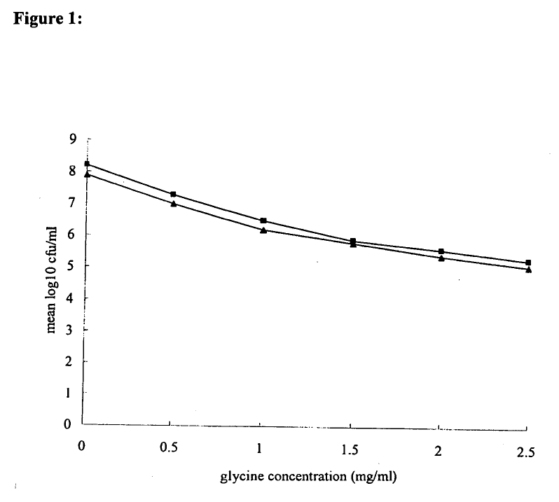

Glycine has been used as a food additive due to its low toxicity in animals (11, 13, 33). For example, Lactococcus lactis subspecies failed to grow in medium containing more than 2% glycine (15). Furthermore, glycine concentrations of 1.5 to 6% resulted in 70 to 90% reductions in growth of Enterococcus faecalis (10). Although glycine is known to inhibit the synthesis of a peptidoglycan component of the bacterial cell wall (16); heretofore, there has been no conclusive evidence to suggest uniformity in the antibacterial efficacy or any antibacterial efficacy of glycine. In fact, a survey of the relevant art reveals that the efficacy of glycine toward H. pylori as found in the present investigation (80% Inh. %=0.05 mg/ml; see FIG. 1) far surpasses that which would be expected. In other words, based on the proposed role for glycine in bacterial cell wall synthesis, it may be expected that the amount of glycine required to suppress gram-negative bacterial growth is lower than that required to suppress gram-positive bacteria because the bacterial cell wall is thinner in gram-negative bacteria than in gram-positive bacteria (30). However, this “expectation” has not proved to be true as a survey of the art represented by references (13), (15), (16), and (41) reveal.

AMX is a beta-lactam antibiotic which inhibits the synthesis of a peptidoglycan of the cell wall, is also effective against H. pylori (6), albeit with an eradication rate when it is used alone that is lower than those of the other drugs used in combination (23), and AMX-resistant H. pylori are rare (1). The study presented in the Examples was conducted to determine the effect of glycine on H. pylori, especially CLR-resistant strains, and to evaluate the PAE of glycine and the effect of glycine in combination with AMX. The results below indicate that administration of glycine in conjunction with AMX represent a new therapy for H. pylori eradication.

The inhibition of proliferation of H. pylori was dependent on the glycine concentration. Furthermore, the MBC of AMX decreased to 1/10 when it was used together with glycine, suggesting that glycine may have a synergistic effect with AMX. Cell wall synthesis involves a number of available active enzymes, such as DD- and DL-carboxypeptidases, which are important for the formation of cell wall-bound peptidoglycan. Because glycine inhibits LD-carboxypeptidase, the modifying effect of glycine on cell wall synthesis was explained to be largely due to this mechanism. Beta-lactam antibiotics are capable of inhibiting not only DL-carboxypeptidases but also DDcarboxypeptidases, which are required for the synthesis of cross-linked peptidoglycan in Gaffkya homari (14). Given these observations, we assume that the synergism of glycine in increasing the antimicrobial efficacies of beta-lactam antibiotics is mainly due to its inhibition of this enzyme system (11). However, H. pylori murein lacks LD-cross-linked-carboxypeptidase muropeptides, and H. pylori may not have LD-carboxypeptidases (22). Therefore, an alternative is that the synergism of AMX and glycine against H. pylori involves AMX inhibition of DL- or DD-carboxypeptidases and glycine mainly inhibits UDPN-acetylmuramate-alanine ligase.

In view of the foregoing, the present invention provides a pharmaceutical compositions containing a therapeutically effective amount of glycine or a pharmaceutically acceptable salt thereof; and a therapeutically effective amount of at least one additional Helicobacter pylori therapeutic agent. The composition of the present invention further contains a pharmaceutically acceptable carrier(s) or excipient(s).

To this end, and as explained further below, the additional Helicobacter pylori therapeutic agent(s) may be, in any combination, amoxicillin, clarithromycin, a proton pump inhibitor, and a histamine-H2 receptor blocking compound.

In another embodiment of the present invention is a method for the prophylactic and/or therapeutic treatment of at least one disorder induced, caused or mediated by Helicobacter pylori infection comprising: administrating to a subject in need thereof a therapeutically effective amount of glycine or a pharmaceutically acceptable salt thereof.

In a further embodiment, the Helicobacter pylori is clarithromycin-resistant Helicobacter pylori.

In still further embodiment, the disorder induced, caused or mediated by Helicobacter pylori infection is preferably a peptic ulcer, malignant lymphoma, gastric cancer, chronic urticaria, and thrombocytopenia.

As used in the present application, the term “subject in need thereof” is used to designate the subject as being one with a recognized need for prophylactic and/or therapeutic treatment of at least one disorder induced, caused or mediated by Helicobacter pylori infection. Further, it should be noted that the “subject” may be human or of animal (i.e., non-human origin), including canine (e.g., dogs, etc.), feline (e.g., cats, etc.), rodentia (e.g., mice, rats, etc.), equine (e.g., horses, etc.), and swine (e.g., pigs, etc).

Within the methods of the present invention, the mode of administering is preferably by a mode of administration selected from the group consisting of oral administration, parenteral administration, and intraperioneal administration; however, additional modes of administration as explained below are also within the scope of the preset invention.

Further, in the methods of the present invention, the therapeutically effective amount ranges from 500 mg to 30 g of glycine or a pharmaceutically acceptable salt thereof administered one to three times per day (see below for a more detailed discussion of the preferred ranges.

In yet another embodiment of the present invention is to provide a method for the prophylactic and/or therapeutic treatment of at least one disorder induced, caused or mediated by Helicobacter pylori infection by administrating to a subject in need thereof a therapeutically effective amount of glycine or a pharmaceutically acceptable salt thereof; and at least one additional Helicobacter pylori therapeutic agent.

Within this embodiment, (a) and (b) may be concurrently administered or sequentially administered. Additionally, (a) and (b) may also be individually or collectively combined with one or more additional Helicobacter pylori therapeutic agent, such as a proton pump inhibitor, and a histamine-H2 receptor blocking compound.

In an embodiment of this object, the Helicobacter pylori is clarithromycin-resistant Helicobacter pylori.

In an embodiment of the present invention, in the aforementioned method said additional Helicobacter pylori therapeutic agent is amoxicillin. When said additional Helicobacter pylori therapeutic agent is amoxicillin the therapeutically effective amount ranges from 500 mg to 30 g of glycine or a pharmaceutically acceptable salt thereof and from 25 mg to 750 mg of amoxicillin administered one to three times per day. Further, said additional Helicobacter pylori therapeutic agent may be clarithromycin, a proton pump inhibitor, and/or a histamine-H2 receptor blocking compound.

Thus, in yet another embodiment of the present invention is to provide a method for the prophylactic and/or therapeutic treatment of at least one disorder induced, caused or mediated by Helicobacter pylori infection by administering a therapeutically effective amount of glycine or a pharmaceutically acceptable salt thereof; a therapeutically effective amount of amoxicillin and a therapeutically effective amount of clarithromycin; and a therapeutically effective amount of a proton pump inhibitor.

As used herein the term “CFU” means colony forming unit, “MIC” means minimum inhibitory concentration, and “MBC” means Minimum Bactericidal concentration.

One skilled in the art is familiar with numerous methods for designing and optimizing formulations and delivery methods to deliver the compositions of the present invention, which contain glycine or a pharmaceutically acceptable salt thereof in effective and non-toxic ways. Remington's Pharmaceuticals Sciences, 18th Edition (specifically incorporated herein by reference), can be relied on and used for these purposes, especially Part 8 therein, “Pharmaceutical Preparations and Their Manufacture.” The following compounds, compositions, delivery methods, delivery dosages, and formulations are specifically envisioned as suitable for, but not meant to limit, the present invention.

The pharmaceutical compounds suitable for administration in the present invention may be hydrochloride salts, but the free bases and other pharmaceutically acceptable salts are also suitable.

Further, the term “pharmaceutically acceptable salt” is well known in the art, as described in S. M. Berge, et al. (J Pharmaceutical Sciences, 66: 1-19, 1977). Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably nontoxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, furnarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, lower alkyl sulfonate and aryl sulfonate.

A therapeutically effective amount of the pharmaceutical compounds suitable for administration in the present invention may be administered alone or in combination with one or more pharmaceutically acceptable carriers. As used herein, the term “pharmaceutically acceptable carrier” means a non-toxic, inert solid, semi-solid or liquid filer, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions suitable for administration in the invention can be administered to patients in need thereof orally, rectally, nasally, parenterally (e.g., intramuscular, intraperitoneal, intravenous or subcutaneous injection, or implant), intracisternally, intravaginally, intraperitoneally, sublingually, topically (e.g., as a powder, ointment, or drop), bucally, as an oral spray, or a nasal spray. The pharmaceutical compositions can be formulated in dosage forms appropriate for each route of administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art. The inert diluents may include, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The liquid dosage form for oral administration may also contain adjuvants, which include wetting agents, emulsifying and suspending agents, sweetening, flavoring, and perfuming agents. Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example safflower oil, coconut oil, corn oil, cottonseed oil, sunflower seed oil, canola oil, soybean oil, and hemp seed oil.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug (in this case glycine or a pharmaceutically acceptable salt thereof), it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, dissolving or suspending the drug in an oil vehicle accomplishes delayed absorption of a parenterally administered drug form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, prills, powders, and granules. In such solid dosage forms, the active compound is mixed with at least one inert, pharmaceutically acceptable excipient or carrier. In addition, the solid dosage form may contain one or more fillers, extenders, binders, humectants, disintegrating agents, retarding agents, absorption accelerators, wetting agents, absorbents, or lubricants. Examples of suitable fillers or extenders include, starches, lactose, sucrose, glucose, mannitol, and silicic acid, sodium citrate and dicalcium phosphate. Examples of suitable binders include, microcrystalline cellulose, carboxymethylcellulose, alginates, gelatin, polyvinylpyrrolidinone, sucrose, and acacia. Glycerol is an example of a suitable humectant. Examples of suitable disintegrating agents include, agar-agar, calcium carbonate, potato or tapioca starch, maize starch, alginic acid, certain silicates, and sodium carbonate. Paraffin is an example of a suitable solution-retarding agent. As absorption accelerators, any quaternary ammonium compound may be used. Examples of suitable wetting agents include, cetyl alcohol and glycerol monostearate. Examples of suitable absorbents include, kaolin and bentonite clay. Examples of suitable lubricants include, talc, calcium stearate, magnesium stearate, solid polyethylene glycols, sodium lauryl sulfate. In the case of capsules, tablets and pills, the dosage form may also comprise buffering agents.

The tablets may, if desired, be coated using known methods and excipients that may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hardfilled gelatin capsules using such excipients as lactose or milk sugar as well as high molecular weight polyethylene glycols.

If desired, the compounds of the present invention can be incorporated into slow release or targeted delivery systems such as polymer matrices, liposomes and microspheres. They may be sterilized, for example, by filtration through a bacteria-retaining filter, or by incorporating sterilizing agents in the form of sterile solid compositions that can dissolve in sterile water, or some other sterile injectable medium immediately before use.

The active compound may be formulated into granules with or without additional excipients. The granules may be ingested directly by the patient or they may be added to a suitable liquid carrier (for example, water) before ingestion. The granules may contain disintegrates, e.g. an effervescent couple formed from an acid and a carbonate or bicarbonate salt to facilitate dispersion in the liquid medium.

Dosage forms for topical or transdermal administration of a compound of this invention include ointments, pastes, creams, lotions, gels, powders, solutions, sprays, inhalants or patches. Transdermal patches have the added advantage of providing controlled delivery of a compound to the body. The rate can be controlled by either providing a rate controlling membrane or by dispersing the compound in a polymer matrix or gel. The active component is admixed under sterile conditions with a pharmaceutically acceptable carrier and any needed preservatives or buffers as may be required. Dissolving or dispensing the compound in the proper medium can make such dosage forms. Absorption enhancers can also be used to increase the flux of the compound across the skin. Ophthalmic formulation, eardrops, eye ointments, powders and solutions are also contemplated as being within the scope of this invention.

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as animal and vegetable fats, oils, petrolatum, waxes, paraffins, starch, tragacanth, cellulose derivatives, polyethylene glycols, silicones, bentonites, silicic acid, talc and zinc oxide, or mixtures thereof, together with a potential transdermal accelerant such as dimethyl sulphoxide or propylene glycol. Alternatively the active compounds may be dispersed in a pharmaceutically acceptable paste, cream, gel or ointment base. The amount of active compound contained in a topical formulation should be such that a therapeutically effective amount of the compound is delivered during the period of time for which the topical formulation is intended to be on the skin.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. The therapeutically active compound may be formulated into a composition, which is dispersed as an aerosol into the patient's oral or nasal cavity. Such aerosols may be administered from a pump pack or from a pressurized pack containing a volatile propellant.

In a preferred embodiment of the present invention, the pharmaceutical composition containing glycine or a pharmaceutically acceptable salt thereof may be prepared as a food or dietary supplement. In this regard, the pharmaceutical composition may be admixed directly with a food or beverage in a therapeutically acceptable amount. To this end, the therapeutically acceptable amount is 500 to 90,000 mg/kg of said food, preferably 2500 to 18,000 mg/kg of said food or 0.5 to 90 mg/ml of said beverage, preferably 2.5 to 18 mg/ml of said beverage.

The therapeutically active compounds used in the method of the present invention (i.e., glycine or a pharmaceutically acceptable salt thereof) may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

Within the context of the present invention, speaking in terms of the active ingredients, glycine or the pharmaceutically acceptable salt thereof may be administered independently or in the presence of one or more additional active therapeutic agents (also referred to herein as additional antibacterial compound(s)) for the treatment, prevention, amelioration, or prophylaxis of diseases induced by, caused by, and/or mediated by H. pylori infection. To this end, the skilled artisan would appreciate that there are numerous such compounds, which may be used for the above-stated purposes in relation to H. pylori infection. Combinations of these therapeutic agents, some of which have also been mentioned herein, will bring additional, complementary, and often synergistic properties to enhance the desirable properties of these various therapeutic agents.

In these combinations, the glycine or the pharmaceutically acceptable salt thereof and the therapeutic agents may be independently present in dose ranges from one one-hundredth to one times the dose levels which are effective when these compounds are used singly. In such combination therapy, the glycine or the pharmaceutically acceptable salt thereof may be administered with the other therapeutic agent (e.g., concurrently, concomitantly, sequentially, or in a unitary formulation) such that their therapeutic efficacy overlaps.

To this end, it is preferable that the additional H. pylori therapeutic agents that may be employed in conjunction with glycine or the pharmaceutically acceptable salt thereof an agent selected from the group consisting of amoxicillin; clarithromycin; one or more proton pump inhibitors, which include: omeprazole, esomeprazole, lansoprazole, leminoprazole, rabeprazole, and nepaprazole; and one or more histamine-H2 receptor blocking compounds, which include: ranitidine, cimetidine, and famotidine. Of course, the present invention also embraced pharmaceutically acceptable salts of the aforementioned additional therapeutic agents.

As used herein, the term “therapeutically effective amount” refers to that amount of a compound or preparation of the present invention that successfully treats, prevents, and/or ameliorates a disease(s) induced by, caused by, and/or mediated by H. pylori infection. This term also embraces the amount of a compound or preparation of the present invention that successfully prevents or reduces the severity of symptoms associated with a disease(s) induced by, caused by, and/or mediated by H. pylori infection. To this end, the disorders that may be induced by, caused by, and/or mediated by H. pylori preferably include peptic ulcer (including stomach ulcer and duodenum ulcer), malignant lymphoma, gastric cancer, chronic urticaria, and thrombocytopenia.

It is contemplated that the therapeutically effective amount of a composition will depend on a number of factors, including by not limited to the age of the patient, immune status, race, and sex of the patient, and the severity of the condition/disease, and the past medical history of the patient, and always lies within the sound discretion of the administering physician. Generally, the glycine or a pharmaceutically acceptable salt thereof is administered to a patient in dosage form containing from 500 mg to 30 g of glycine, preferably 1 g to 15 g of glycine, more preferably 2.5 g to 6 g of glycine.

In one embodiment of the present invention to administer the above-recited dosage one, two, or three times daily.

Thus, in one embodiment of the present invention, glycine or a pharmaceutically acceptable salt is administered in a total daily dosage ranging from 500 mg to 90 g per day, preferably 1 g to 45 g per day, more preferably 2.5 g to 18 g per day.

In another embodiment of the present invention, glycine or a pharmaceutically acceptable salt thereof is administered to a patient in single or in divided doses can be in amounts, for example, 10 to 1800 mg/kg of body weight/day of glycine (approximately 500 mg to 90 g/day), preferably 20 to 900 mg/kg of body weight/day of glycine (approximately 1 to 45 g/day), and most preferably 50 to 360 mg/kg of body weight/day of glycine (approximately 2.5 to 18 g/day). Single dose compositions may contain such amounts or submultiples thereof to make up the daily dose.

Amoxicillin-

When amoxicillin is used in combination with glycine, it is preferred that the ratio of glycine to amoxicillin range from 1.25:1 to 5000:1, preferably 2.5:1 to 2500:1, more preferably 10:1 to 1000:1.

Further, amoxicillin, when present, may be administered to a patient in dosage form containing from 20 to 5000 mg of amoxicillin, preferably 40 to 2500 mg of amoxicillin, more preferably 100 to 1000 mg of amoxicillin.

In one embodiment of the present invention to administer the above-recited dosage one, two, or three times daily.

Thus, in one embodiment of the present invention, amoxicillin is administered in a total daily dosage ranging from 20 to 15,000 mg per day, preferably 40 to 7500 mg per day, more preferably 100 to 3000 mg per day.

In an embodiment of the present invention, amoxicillin is administered as a 1000 mg (±25%) dosage form. In the embodiment, this dosage form is administered twice daily. Further, within this embodiment, this dosage form is in admixture with one or more of clarithromycin, a proton pump inhibitor, and a histamine-H2 receptor blocking compound.

Clarithromycin-

When clarithromycin is used in combination with glycine, it is preferred that the ratio of glycine to clarithromycin range from 1.25:1 to 5000:1, preferably 2.5:1 to 2500:1, more preferably 10:1 to 1000:1.

Further, clarithromycin, when present, may be administered to a patient in dosage form containing from 20 to 5000 mg of clarithromycin, preferably 40 to 2500 mg of clarithromycin, more preferably 10 to 300 mg of clarithromycin.

In one embodiment of the present invention to administer the above-recited dosage one, two, or three times daily.

Thus, in one embodiment of the present invention, clarithromycin is administered in a total daily dosage ranging from 20 to 15,000 mg per day, preferably 40 to 7500 mg per day, more preferably 100 to 3000 mg per day.

In an embodiment of the present invention, clarithromycin is administered as either a 500 mg (±25%) dosage form or a 250 mg (±25%) dosage form. In the embodiment, this dosage form is administered twice daily. Further, within this embodiment, this dosage form is in admixture with one or more of amoxicillin, a proton pump inhibitor, and a histamine-H2 receptor blocking compound.

Proton Pump Inhibitors-

When a proton pump inhibitor is used in combination with glycine, it is preferred that the ratio of glycine to proton pump inhibitor range from 1.25:1 to 5000:1, preferably 2.5:1 to 2500:1, more preferably 10:1 to 1000:1.

Further, a proton pump inhibitor, when present, may be administered to a patient in dosage form containing from 2 to 1500 mg of proton pump inhibitor, preferably 4 to 750 mg of proton pump inhibitor, more preferably 10 to 300 mg of proton pump inhibitor.

In one embodiment of the present invention to administer the above-recited dosage one, two, or three times daily.

Thus, in one embodiment of the present invention, a proton pump inhibitor is administered in a total daily dosage ranging from 2 to 4500 mg per day, preferably 4 to 2250 mg per day, more preferably 10 to 900 mg per day.

In an embodiment of the present invention, a proton pump inhibitor is administered as a 30 mg (±25%) dosage form. In the embodiment, this dosage form is administered twice daily. Further, within this embodiment, this dosage form is in admixture with one or more of amoxicillin, clarithromycin, and a histamine-H2 receptor blocking compound.

Histamine-H2 Receptor Blocking Compounds-

When a histamine-H2 receptor blocking compound is used in combination with glycine, it is preferred that the ratio of glycine to histamine-H2 receptor blocking compound range from 1.25:1 to 5000:1, preferably 2.5:1 to 2500:1, more preferably 10:1 to 1000:1.

Further, a histamine-H2 receptor blocking compound, when present, may be administered to a patient in dosage form containing from 2 to 1500 mg of histamine-H2 receptor blocking compound, preferably 4 to 750 mg of histamine-H2 receptor blocking compound, more preferably 10 to 300 mg of histamine-H2 receptor blocking compound.

In one embodiment of the present invention to administer the above-recited dosage one, two, or three times daily.

Thus, in one embodiment of the present invention, a histamine-H2 receptor blocking compound is administered in a total daily dosage ranging from 2 to 4500 mg per day, preferably 4 to 2250 mg per day, more preferably 10 to 900 mg per day.

In a particularly preferred embodiment, a histamine-H2 receptor blocking compound is administered twice daily. Further, within this embodiment, this dosage form is in admixture with one or more of amoxicillin, clarithromycin, and a proton pump inhibitor.

Further, the submultiples of the aforementioned dosage forms may be used as part of a treatment regimen in which single or multiple doses are administered daily to maximum daily dose for a defined time. Treatment regimens according to the present invention also include concurrently administering to a patient in need thereof mixtures, in single or divided doses, of two or more of the compounds of the present invention. When the compounds of the present invention are administered concurrently as mixtures, the therapeutically effective amount to be administered lies within the sound discretion of the administering physician.

Alternatively, treatment regimens according to the present invention include sequentially administering to a patient in need thereof, in single or divided doses, two or more of the compounds of the present invention (i.e., glycine and one or more additional therapeutic agents). An example of a sequential administration strategy includes administering a therapeutically effective amount of a first compound followed by, on the same day or a subsequent day, a single or divided dose of a therapeutically effective amount of one or more additional compounds. As used herein, the term “subsequent day” refers to any day ranging from the next day (>24 hours) to one week (≦168 hours) after administration of the previous compound. The term “same day” refers to any time frame ranging from immediately after administration of the previous compound to ≦24 hours after administration of the previous compound.

When the compounds of the present invention are administered sequentially as a part of a combination therapy, the therapeutically effective amount to be administered lies within the sound discretion of the administering physician.

Within the scope of the present invention, the present inventors envision the use of glycine as part of a combination therapy in conjunction with one or more one or more of amoxicillin, clarithromycin, a proton pump inhibitor, and a histamine-H2 receptor blocking compound. In this embodiment, it is expected that the dosage ranges for use thereof are to be adjusted based on clinical indication and dose response, as well as safety and efficacy concerns of the compounds used therein. Therefore, it is envisioned that the dosage ranges recited above for each of the compounds recited for use in said combination therapy may be reduced by as much as 10%, preferably as much as 25%, more preferably as much as 50%.

In an embodiment the present invention, glycine (at a dose listed above) is administered in conjunction with (either sequentially or simultaneously) the following dosage form:

1000 mg of amoxicillin;

either 250 mg or 500 mg of clarithromycin; and

30 mg of a proton pump inhibitor.

The dosage form above may further contain a histamine-H2 receptor blocking compound. In the embodiment, the dosage form is administered twice daily. However, this dose form is exemplary only. Within the present invention the above-recited quantities may be altered by ±25% and still fall within the scope of the present invention.

As used herein, the terms “treat”, “treating”, and “treatment” also embrace the terms alleviation and amelioration. In addition, it is also within the scope of the present invention to use the methods described and/or claimed herein for the treatment or prevention of a disease(s) induced by, caused by, and/or mediated by H. pylori infection, as well as the symptoms associated therewith. Moreover, the term “prevention” embraces prophylaxis.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Materials and Methods-

Agents:

Glycine (assay [after drying] minimum, 99.0%; soluble in water; maximum amount of chloride, 0.02%, maximum amount of sulfate, 0.005%; maximum amount of heavy metal [as Pb], 0.001%; maximum amount of iron, 0.0005%; maximum amount of ammonium, 0.02%; total nitrogen, 18.5 to 18.8%; Wako Pure Chemical Industries, Ltd., Osaka, Japan) and AMX (Sigma Chemical Co., St. Louis, Mo.) were purchased, and CLR (Taisho Pharmaceutical Co., Ltd., Osaka, Japan) was kindly provided as a gift. Glycine, CLR, and AMX were dissolved in distilled water to a final concentration of 20% (wt/vol), methanol, and 70% ethanol, respectively.

Bacterial Strains and Culture Conditions:

The susceptibilities to glycine of 31 H. pylori strains, including three standard strains (strains 26695 [37] and J99 [2] and a quality control strain [ATCC 43504] [27]), and clinical isolates (4, 28, 35, 38) were measured. The clinical isolates were from biopsy specimens of lesions obtained by endoscopy from different patients with gastroenterological diseases at Nagoya University Hospital and a regional health care center. Stock cultures of H. pylori were grown for 4 days on brucella broth agar (Difco, Detroit, Mich.) plates supplemented with 7% heat-inactivated fetal calf serum (FCS; Gibco BRL, Rockville, Md.) (BB agar) at 37° C. in a microaerophilic atmosphere. Broth cultures of H. pylori were prepared from subcultures of colonies from freshly cultured agar plates placed into brucella broth (Difco) supplemented with 7% FCS (BB) for 3 days at 37° C. in a microaerophilic atmosphere. The identification of H. pylori was confirmed by characteristic colony morphology, Gram staining, and positive reactions by urease, catalase and oxidase tests.

DNA Techniques:

Standard molecular biology-based techniques were used (29). H. pylori chromosomal DNA was prepared from cells of each strain after 48 h of growth on two agar plates and was extracted with a Wizard Genomic DNA Purification kit (Promega, Madison, Wis.), according to the instructions of the manufacturer.

Construction of Isogenic Mutant Strains:

CLR-resistant isogenic mutants were constructed by natural transformation methods (3, 29). Domain V of the 23S rRNA gene in H. pylori, which is associated with CLR resistance (32), was amplified by PCR. Template DNA was extracted from CLR-resistant strains 628 and 535 and was found to possess A-to-G point mutations at either position 2143 (strain 628) or position 2144 (strain 535) in domain V of the 23S rRNA gene.

The PCR products from donor DNA samples were purified. Briefly, 1 ng of DNA was amplified with 100 pmol of the sense and antisense primers (sense primer, 5′-CCACAGCGATGTGGTCTCAG-3′ [positions 1820 to 1839 of 23S rRNA sequence; SEQ ID NO: 1]; antisense primer, 5′-CTCCATAAGAGCCAAAGCCC-3′ [positions 2244 to 2225 of the 23S rRNA sequence; SEQ ID NO: 2]) in a 50-μl reaction mixture containing 0.25 μl of Ex Taq polymerase (Takara Biomedicals, Ohtsu, Japan) for 25 cycles of 1 min at 94° C., 1 min at 55° C., and 1 min at 72° C. in a DNA thermal cycler (GeneAmp PCR System 9700; Applied Biosystems, Foster City, Calif.). The presence of a single 425-bp band was verified on a 1% agarose gel, and the PCR products were purified with a QIAquick PCR Purification kit (Qiagen, Valencia, Calif.).

H. pylori strain 26695 was used as the transformation recipient. After 48 h of growth, recipient H. pylori cells were harvested from one BB agar plate and placed into 1 ml of phosphate-buffered saline (PBS) and then centrifuged at 8,500×g for 5 min. The pellet was resuspended in 300 μl of PBS. Each transformation mixture, consisting of 25 μl of recipient cells and 1 μg of donor DNA, was spotted onto a BB agar plate (approximately 600 ng of DNA/25 μl of cells is a saturating amount of DNA). The plates were incubated overnight at 37° C. in a microaerophilic atmosphere. After 18 h of incubation, the transformation mixture was spread onto BB agar containing 4 μg of CLR/ml. All plates were incubated for 5 days at 37° C. in a microaerophilic atmosphere to select transformants (3). The presence of point mutations in the transformants was confirmed by PCR and direct sequencing (CEQ2000XL; Beckman Coulter Inc., Fullerton, Calif.).

Susceptibility Testing:

The susceptibilities of the CLR-resistant strains were determined by the agar dilution method (27). An MIC of more than 1 μg/ml was considered CLR resistance. The MIC of glycine was defined as the lowest concentration that inhibited the visible growth of isolates completely by the agar dilution method on Mueller-Hinton agar (Becton Dickinson and Company, Paramus, N.J.) plates supplemented with 5% aged sheep blood (Nippon Bio-Test Laboratories Inc., Tokyo, Japan) (M-H agar) (27).

Briefly, all isolates were incubated for 4 days on BB agar. After this incubation, inocula were prepared by suspending growth from the BB agar plates with antimicrobial agents in saline to achieve a suspension equivalent to a 2.0 McFarland standard. Final inocula of 10⁶ CFU/spot were applied to M-H agar or BB. All plates were incubated for 3 days at 35° C. in a microaerophilic atmosphere, and the number of CFU was counted. In the liquid culture study, aliquots from each culture were applied to BB agar after 3 days of incubation of H. pylori at 37° C. under microaerophilic conditions. After 4 days of incubation, the number of CFU was counted. The mean value for the log number of CFU per milliliter was plotted.

By a second method, the minimal bactericidal concentrations (MBCs), defined as the lowest concentration of each antibiotic that completely killed the isolates, were determined by using liquid cultures with antimicrobial agents by a modification of the method of Sjöström et al. (31). The MBC of AMX with glycine was determined by serial dilution of AMX and glycine in BB in the presence of approximately 10⁶ CFU/ml in 25-ml tissue culture flasks (Becton Dickinson and Company). After 3 days of incubation of H. pylori at 37° C. under microaerophilic conditions, aliquots from each culture were applied to BB agar for determination of the MBC. After 4 days of incubation, the number of CFU was counted. AMX and glycine were tested at concentrations ranging from 0 to 75 ng/ml and 0 to 2.5 mg/ml, respectively. The MBCs were measured to study the effect of the interaction between AMX and glycine against strain 26695.

Determination of PAE:

The PAE on bacteria was measured by a modification of the method of Ibrahim et al. (19). After 4 days of growth of strain 26695 on BB agar, the bacteria were harvested, placed into PBS, and diluted with BB to ˜10⁸ CFU/ml; and a final inoculum of 10⁶ CFU/ml was applied to BB agar. Glycine was added to BB in a 25-ml tissue culture flask at a concentration of 1.25 mg/ml (five times the MIC). After incubation for 8 h, the antibiotics were removed by dilution 1:10³ into fresh BB and the cultures were incubated for 24 h. Samples were collected for viable counts (numbers of CFU per milliliter) every 4 h, plated onto BB agar plates, and incubated for 4 days. Viability curves were then determined. The control culture, which was not exposed to any antimicrobial agents, was treated similarly.

For quantification of the PAE, viable counts were determined before and after antibiotic exposure. The T−C value was calculated as follows (8): T was the time required for the CFU count in the test culture to increase 10-fold above the count observed immediately after antibiotic removal, and C was the time required for the CFU count in an untreated control culture to increase 10-fold above the count observed immediately after completion of the same procedure for antibiotic removal used for the test culture.

Time-Kill Curve Test:

The time course of the antibacterial effects was determined with strain 26695 (18). Briefly, bacteria cultured for 4 days on BB agar were diluted with fresh BB to ˜10⁸ CFU/ml. A final inoculum of 10⁶ CFU/ml was placed into a 25-ml tissue culture flask containing 10 ml of BB with glycine or both glycine and AMX. Broth cultures with glycine were incubated for 2 days at 37° C. in a microaerophilic atmosphere. At 0, 12, 24, 36, and 48 h, samples were removed and 0.1 ml of 10-fold serial dilution was plated onto BB agar. Broth cultures with both glycine and AMX were incubated for 1 day at 37° C. in a microaerophilic atmosphere. At 0, 6, 12, 18, and 24 h, samples were removed and 0.1 ml of 10-fold serial dilutions was plated onto BB agar. The number of colonies growing on the plates after 4 days of incubation was counted in both studies.

Statistical Analysis:

The degree of significance between means was determined by the paired t test. A P value of <0.01 was regarded as significant. These studies were repeated at least five times to confirm the reliability of the data.

Results-

Construction of CLR-Resistant Isogenic Mutants and MIC Determination:

CLR-resistant isogenic mutants with a single point mutation in domain V of the 23S rRNA gene were created from strain 26695 by natural transformation (3, 29). Transformants were isolated, and the MICs of CLR were determined by the disk dilution method. Strain 26695:628 possessed an A-to-G point mutation at position 2143 in domain V of the 23S rRNA gene, and strain 26695:535 possessed an A-to-G point mutation at position 2144. The MIC of CLR was dependent on that for the donor strain (for strain 628, MIC=32 μg/ml; for strain 535, MIC=4 μg/ml; for strain 26695:628; MIC=32 μg/ml; for strain 26695:535, MIC=4 μg/ml).

Glycine MICs:

The effect of glycine on 31 bacterial strains, including 10 CLR-resistant H. pylori strains, was examined. The glycine MICs for the 31 strains ranged from 1 to 2.5 mg/ml, while the concentrations required to inhibit 50 and 90% of the strains (MIC₅₀ and MIC₉₀, respectively) were 1.5 and 2.5 mg/ml, respectively. Glycine inhibited the growth of all strains tested, and no colonies were detected in M-H agar with glycine at more than 2.5 mg/ml. The MIC of glycine for the quality control strain (ATCC 43504) was same as that for strain 26695 (2.5 mg/ml). There was no difference in the glycine MIC for CLR-susceptible and -resistant strains. Furthermore, there was no association between the MIC of CLR and the MIC of glycine. The MIC of glycine for the experimental strain was dependent on that for the recipient strain (strain 26695), and the MICs for the isogenic mutant strains were the same as the MIC for strain 26695.

These data strongly suggest that the antibacterial mechanisms of glycine and CLR are independent. The addition of glycine resulted in a significant decrease in the number of bacteria. Furthermore, significant concentration-dependent suppression of bacterial growth was observed for both strains. However, there was no difference in the suppression of growth between the CLR-susceptible and -resistant strains, including the isogenic mutant strains (strains 26695:628 and 26695:535) (See FIG. 1). In other words, glycine acted similarly against both CLR-resistant and -susceptible H. pylori.

Time-Kill Curve Experiments with Glycine:

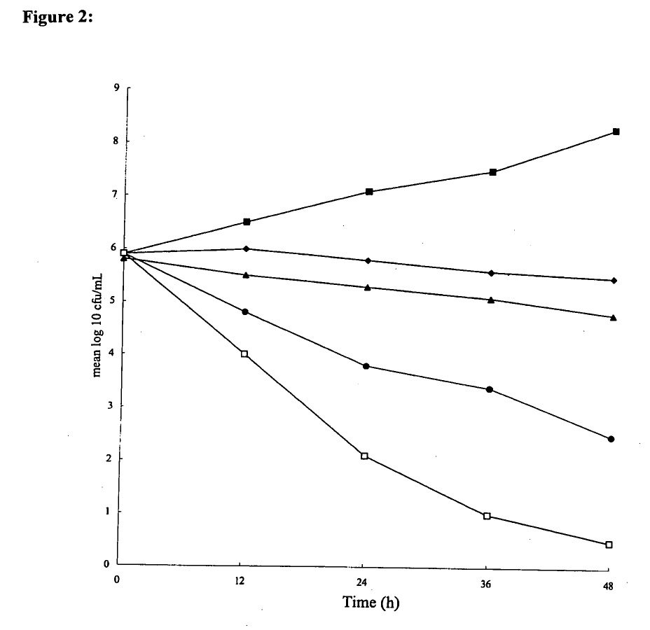

The results of the time-kill curve experiments with glycine alone at a concentration of 0 to 10 mg/ml are shown in FIG. 2. The number of CFU increased over time when no glycine was present in the culture medium. When glycine was present, however, a significant decrease in the number of CFU was seen at 48 h with all concentrations (P<0.01). This decrease was significant (P<0.01) at as early as 24 h with a concentration of 5 mg/ml and as early as 12 h with a concentration of 10 mg/ml. These results show that the effect of glycine is bactericidal, because the number of CFU was not stable but was decreased by glycine administration.

Effect of Combination of Glycine with AMX:

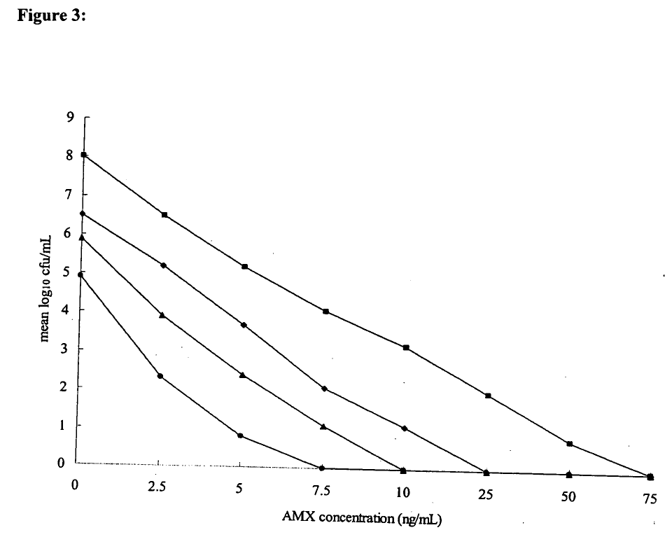

The effect of the combination of glycine with AMX against H. pylori was also determined The MBC of AMX with glycine was determined by the liquid culture method with the following doses: AMX at 7.5 ng/ml with glycine at 2.5 mg/ml, AMX at 10 ng/ml with glycine at 1 mg/ml, AMX at 25 ng/ml with glycine at 0.5 mg/ml, and AMX at 75 ng/ml with glycine at 0 mg/ml (see FIG. 3). The MBC of AMX and glycine in combination was less than that of AMX alone (P<0.01). It was found that the combination of AMX and glycine reduced the viable count for strain 26695, even at concentrations that had subinhibitory effects when the agents were used alone. Their combined effect against other H. pylori strains was the same as that against strain 26695 (data not shown). Incubation with AMX and glycine yielded significant inhibition of growth for strain 26695. The combination of AMX and glycine enhanced the killing effect against strain 26695 and resulted in a dramatic decrease in the number of viable cells that was greater than that achieved with glycine or AMX alone.

Time-Kill Curve Experiments with Glycine and AMX:

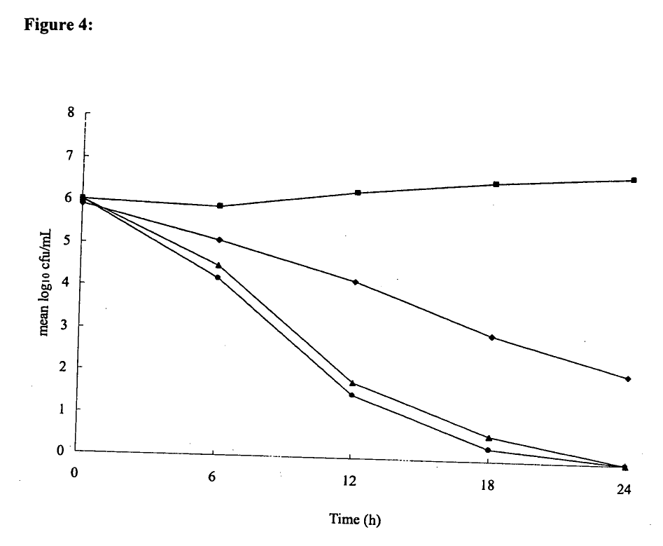

A time-kill curve experiment was performed to investigate the rate at which the viable cell count decreased after treatment with glycine and AMX (see FIG. 4). The number of bacterial colonies increased in the absence of glycine. However, when a combination of glycine at 2.5 mg/ml and AMX at 7.5 ng/ml (the MBCs) was used, a significant decrease in the number of bacteria was seen after 6 h (P<0.01). Moreover, the rate of decrease in cell numbers achieved with the combination of glycine and AMX was greater than that achieved with glycine at 10 mg/ml alone and was as fast as that achieved with AMX at 75 ng/ml alone, with no viable cells found at 24 h when either AMX alone at 75 ng/ml or the combination of glycine and AMX was used. Furthermore, strain 26695 was not detected at 24 h when the other combinations of glycine and AMX were used (AMX at 10 ng/ml plus glycine at 1 mg/ml and AMX at 25 ng/ml plus glycine at 0.5 mg/ml). These findings confirm the synergistic activity of glycine and AMX against H. pylori strain 26695.

PAE of Glycine Against H. Pylori:

The PAE regrowth curves for glycine showed growth rates which, after correction for the dilution rate, were close to the growth rate of the control culture measured for this organism. The T−C value was almost zero (data not shown).

Numerous modifications and variations on the present invention are possible in light of the above teachings. It is, therefore, to be understood that within the scope of the accompanying claims, the invention may be practiced otherwise than as specifically described herein.

### REFERENCES

1. Adamek, R. J., S. Suerbaum, and B. Pffaffenbach. 1998. Primary and acquired Helicobacter pylori resistance to clarithromycin, metronidazole, and amoxicillin. influence on treatment outcome. Am. J. Gastroenterol. 93:386. 389.

2. Alm, R. A., L. S. Ling, D. T. Moir, B. L. King, E. D. Brown, P. C. Doig, D. R. Smith, B. Noonan, B. C. Guild, B. L. deJonge, G. Carmel, P. J. Tummino, A. Caruso, M. Uria-Nickelsen, D. M. Mills, C. Ives, R. Gibson, D. Merberg, S. D. Mills, Q. Jiang, D. E. Taylor, G. F. Vovis, and T. J. Trust. 1999. Genomic-sequence comparison of two unrelated isolates of the human gastric pathogen Helicobacter pylori. Nature 397:176.180.

3. Ando, T., D. A. Israel, K. Kusugami, and M. J. Blaser. 1999. HP0333, a member of the dprA family, is involved in natural transformation in Helicobacter pylori. J. Bacteriol. 181:5572.5580.

4. Ando, T., Q. Xu, M. Torres, K. Kusugami, D. A. Israel, and M. J. Blaser. 2000. Restriction-modification system differences in Helicobacter pylori are a barrier to interstrain plasmid transfer. Mol. Microbiol. 37:1052.1065.

5. Asaka, M., T. Sugiyama, M. Kato, K. Satoh, H. Kawayama, Y. Fukuda, T. Fujioka, T. Takemoto, K. Kimura, T. Shimoyama, K. Shimizu, and S. Kobayashi. 2001. A multi center, double-blind study on triple therapy with lansoprazole, amoxicillin and clarithromycin for eradication of Helicobacter pylori in Japanese peptic ulcer patients. Helicobacter 6:254.261.

6. Bayerdörffer, E., G. Mannes, and A. Sommer. 1992. High dose omeprazole treatment combined with amoxicillin eradicates Helicobacter pylori. Eur. J. Gastroentoerol. Hepatol. 4:697.702.

7. Bayerdoörffer, E., A. Neubauer, B. Rudolph, C. Thiede, N. Lehn, S. Eidt, and M. Stolte. 1995. Regression of primary gastric lymphoma of mucosa-associated lymphoid tissue type after cure of Helicobacter pylori infection. Lancet 345:1591.1594.

8. Craig, W. A., and S. Gudmundsson. 1996. Postantibiotic effect, p. 296.329. In V. Lorian (ed.), Antibiotics in laboratory medicine. The Williams & Wilkins Co., Baltimore, Md.

9. Dunn, B. E., H. Cohen, and M. J. Blaser. 1997. Helicobacter pylori. Clin. Microbiol. Rev.

10:720.741. 10. Gary, M. D., N. L. Linda, and J. L. Donald. 1991. Improved electroporation and cloning vector system for gram-positive bacteria. Appl. Environ. Microbiol. 57:1194.1201.

11. Gillissen, G., M. Schumacher, and M. Breuer-Werle. 1991. Modulation of antimicrobial effects of beta-lactams by amino acids in vitro. Zentbl. Bakteriol. Parasitenkd. Infektkrankh. Hyg. Abt. 1 Orig. 275:223.232.

12. Graham, D. Y. 1991. Helicobacter pylori: its epidemiology and its role in duodenal ulcer disease. J. Gastroenterol. Hepatol. 6:105.113.

13. Hammes, W., K. H. Schleifer, and O. Kandler. 1973. Mode of action of glycine on the biosynthesis of peptidoglycan. J. Bacteriol. 116:1029.1053.

14. Hammes, W. P. 1978. The LD-carboxypeptidase activity in Gaffkya homari. The target of the action of D-amino acids or glycine on the formation of wall-bound peptidoglycan. Eur. J. Biochem. 91:501.515.

15. Helge, H., and F. N. Ingolf. 1989. High-frequency transformation, by electroporation, of Lactococcus lactis subsp. cremoris grown with glycine in osmotically stabilized media. Appl. Environ. Microbiol. 55:3119.3123.

16. Hishinuma, F., K. Izaki, and H. Takahashi. 1969. Effects of glycine and D-amino acids on growth of various microorganism. Agric. Biol. Chem. 33:1577.1586.

17. Hojo, M., H. Miwa, A. Nagahara, and N. Sato. 2001. Pooled analysis on the efficiency of the second-line treatment regimens for Helicobacter pylori infection. Scand. J. Gastroenterol. 36:690.700.

18. Horii, T., T. Kimura, K. Sato-Kawamura, T. Nada, K. Shibayama, and M. Ohta. 1999. Beta-lactamase inhibitors have antibacterial activities against Helicobacter pylori. J. Infect. Chemother. 5:206.207.

19. Ibrahim, J., R. M. Hassan, J. Stark, M. Greenman, and R. Millar. 1998. Absence of a post-antibiotic effect (PAE) of beta lactams against Helicobacter pylori NCTC 11637. J. Antimicrob. Chemother. 42:661.663.

20. Ito, E., and J. L. Strominger. 1962. Enzymatic synthesis of the peptide in bacterial uridine nucleotides. J. Biol. Chem. 237:2689.2703.

21. Kato, M., Y. Yamaoka, R. R. Kim, and M. Asaka. 2000. Regional differences in metronidazole resistance and increasing clarithromycin resistance among Helicobacter pylori isolates from Japan. Antimicrob. Agents Chemother. 44: 2214.2216.

22. Katyssulla, C., B. Gerold, A. Gu\ddot{}nter, G. D. Maria, E. Lars, F. Per, A. D. P. VOL. 48, 2004 EFFECT OF GLYCINE ON HELICOBACTER PYLORI 3787 Miguel, and G. D. P. Francisco. 1999. The morphological transition of Helicobacter pylori cells from spiral to coccoid is preceded by a substantial modification of the cell wall. J. Bacteriol. 181:3710.3715.

23. Labenz, J., E. Gyenes, G. H. Ru\ddot{}hl, and G. Bo\ddot{}rsch. 1993. Omeprazole plus amoxicillin: efficacy of various treatment regimens to eradicate Helicobacter pylori. Am. J. Gastroenterol. 88:491.495.

24. Logan, R. P. H., P. A. Gummett, B. T. Hegarty, M. M. Walker, J. H. Baron, and J. J. Misiewicz. 1992. Clarithromycin and omeprazole for Helicobacter pylori. Lancet 340:239.

25. Majcherczyk, P. A. 1996. The issue of the true post antibiotic effect. J. Antimicrob. Chemother. 37:188.189.

26. Marshall, B. J., and J. R. Warren. 1984. Unidentified curved bacilli in the stomach of patients with gastritis and peptic ulceration. Lancet 8390:1311. 1315.

27. NCCLS. 2003. Performance standards for antimicrobial susceptibility testing: M100-S13. NCCLS, Wayne, Pa.

28. Nobata, K., K. Ina, M. Ohta, K. Kawamura-Sato, T. Tsuzuki, T. Ando, and K. Kusugami. 2002. Lower concentrations of clarithromycin suppress urease activity, motility, and binding to gastric epithelial cells in Helicobacter pylori isolates. Dig. Liver Dis. 34:489.497.

29. Sambrook, J., E. F. Fritsch, and T. Maniatis. 1989. Molecular cloning: a laboratory manual, 2nd ed. Cold Spring Harbor Laboratory Press, Cold Spring Harbor, N.Y.

30. Schwartz, A. C., W. Quecke, and G. Brenschede. 1979. Inhibition by glycine of the catabolic reduction of proline in Clostridium sticklandii: evidence on the regulation of amino acid reduction. Z. Allg. Mikrobiol. 19:211.220.

31. Sjöström, J. E., J. Fryklund, T. Ku\ddot{}hler, and H. Larsson. 1996. In vitro antibacterial activity of omeprazole and its selectivity for Helicobacter spp. are dependent on incubation conditions. Antimicrob. Agents Chemother. 40:621.626.

32. Stone, G. G., D. Shortridge, and J. Versalovic. 1997. A PCR-oligonucleotide ligation assay to determine the prevalence of 23S rRNA gene mutations in clarithromycin-resistant Helicobacter pylori. Antimicrob. Agents Chemother. 41:712.714.

33. Stronminger, J. L., and C. H. Birge. 1965. Nucleotide accumulation induced in Staphylococcus aureus by glycine. J. Bacteriol. 89:1124.1127.

34. Sung, J. J. Y., S. Chung, T. K. W. Ling, M. Y. Yung, V. K. S. Leung, E. K. W. Ng, M. K. K. Li, A. F. B. Cheng, and A. K. C. Li. 1995. Antibacterial treatment of gastric ulcers associated with Helicobacter pylori. N. Engl. J. Med. 332:139.142.

35. Takeuchi, H., M. Shirai, J. K. Akada, M. Tsuda, and T. Nakazawa. 1998. Nucleotide sequence and characterization of cdrA, a cell division related gene of Helicobacter pylori. J. Bacteriol. 180:5263.5268.

36. Teresa, A., D. Diego, P. Nuria, and L. B. Manuel. 2000. Clarithromycin resistance stability in Helicobacter pylori: influence of the MIC and type of mutation in the 23S rRNA. J. Antimicrob. Chemother. 46:613.616.

37. Tomb, J. F., O. White, A. R. Kerlavage, R. A. Clayton, G. G. Sutton, R. D. Fleischmann, K. A. Ketchum, H. P. Klenk, S. Gill, B. A. Dougherty, K. Nelson, J. Quackenbush, L. Zhou, E. F. Kirkness, S. Peterson, B. Loftus, D. Richardson, R. Dodson, H. G. Khalak, A. Glodek, K. McKenney, L. M. Fitzegerald, N. Lee, M. D. Adams, E. K. Hickey, D. E. Berg, J. D. Gocayne, T. R. Utterback, J. D. Peterson, J. M. Kelley, M. D. Cotton, J. M. Weidman, C. Fujii, C. Bowman, L. Watthey, E. Wallin, W. S. Hayes, M. Borodovsky, P. D. Karp, H. O. Smith, C. M. Fraser, and J. C. Venter. 1997. The complete genome sequence of the gastric pathogen Helicobacter pylori. Nature 388: 539.547.

38. Tsuzuki, T., K. Ina, M. Ohta, T. Hasegawa, T. Nagasaka, N. Saburi, M. Ueda, T. Konagaya, H. Kaneko, A. Imada, T. Nishiwaki, K. Nobata, T. Ando, and K. Kusugami. 2002. Clarithromycin increases the release of heat shock protein B from Helicobacter pylori. Aliment. Pharmacol. Ther. 16(Suppl. 2):217.228.

39. Uemura, N., S. Okamoto, S. Yamamoto, N. Matsumura, S. Yamaguchi, M. Yamakido, K. Tanaiyama, N. Sasaki, and R. J. Schlemper. 2001. Helicobacter pylori infection and the development of gastric cancer. N. Engl. J. Med. 345:784.789.

40. Yakoob, J., F. Xuegong, H. Guoling, L. Li, and Z. Zheng. 2001. Antibiotic susceptibility of Helicobacter pylori in the Chinese population. J. Gastroenterol. Hepatol. 16:981.985.

41. Gerberick G F and Castric P A. 1980. In vitro susceptibility of Pseudomonas aeruginosa to carbenicillin, glycine, and ethylenediaminetetraacetic acid combinations. Antimicrob Agents Chemother. 17(4):732-5.

1. A pharmaceutical composition comprising:
(a) a therapeutically effective amount of glycine or a pharmaceutically acceptable salt thereof;
(b) a therapeutically effective amount of at least one additional Helicobacter pylori therapeutic agent;
and a pharmaceutically acceptable carrier or excipient.

2. The pharmaceutical composition according to claim 1, wherein said additional Helicobacter pylori therapeutic agent is amoxicillin.

3. The pharmaceutical composition according to claim 2, wherein the ratio of glycine to amoxicillin ranges from 1.25:1 to 5000:1.

4. The pharmaceutical composition according to claim 2, wherein said additional Helicobacter pylori therapeutic agent is clarithromycin.

5. The pharmaceutical composition according to claim 4, further comprising:
(c) a therapeutically effective amount of a proton pump inhibitor.

6. The pharmaceutical composition according to claim 5, further comprising:
(d) a therapeutically effective amount of a histamine-H2 receptor blocking compound.

7. The pharmaceutical composition according to claim 6, wherein said histamine-H2 receptor blocking compound is a member selected from the group consisting of ranitidine, cimetidine, and famotidine.

8. The pharmaceutical composition according to claim 5, wherein said proton pump inhibitor is at least one member selected from the group consisting of omeprazole, esomeprazole, lansoprazole, leminoprazole, rabeprazole, and nepaprazole.

9. The pharmaceutical composition according to claim 4, further comprising:
(d) a therapeutically effective amount of a histamine-H2 receptor blocking compound.

10. The pharmaceutical composition according to claim 9, wherein said histamine-H2 receptor blocking compound is a member selected from the group consisting of ranitidine, cimetidine, and famotidine.

11. The pharmaceutical composition according to claim 2, further comprising:
(c) a therapeutically effective amount of a proton pump inhibitor.

12. The pharmaceutical composition according to claim 2, further comprising:
(d) a therapeutically effective amount of a histamine-H2 receptor blocking compound.

13. The pharmaceutical composition according to claim 1, wherein said additional Helicobacter pylori therapeutic agent is clarithromycin.

14. The pharmaceutical composition according to claim 13, further comprising:
(c) a therapeutically effective amount of a proton pump inhibitor.

15. The pharmaceutical composition according to claim 13, further comprising:
(d) a therapeutically effective amount of a histamine-H2 receptor blocking compound.

16. A method for the prophylactic and/or therapeutic treatment of at least one disorder induced, caused or mediated by Helicobacter pylori infection comprising: administrating to a subject in need thereof a therapeutically effective amount of glycine or a pharmaceutically acceptable salt thereof.

17. The method according to claim 16, wherein said Helicobacter pylori is clarithromycin-resistant Helicobacter pylori.

18. The method according to claim 16, wherein said disorder is selected from the group consisting of peptic ulcer, malignant lymphoma, gastric cancer, chronic urticaria, and thrombocytopenia.

19. The method according to claim 16, wherein said administering is by a mode of administration selected from the group consisting of oral administration, parenteral administration, and intraperioneal administration.

20. The method according to claim 16, wherein said therapeutically effective amount ranges from 500 mg to 30 g of glycine or a pharmaceutically acceptable salt thereof administered one to three times per day.

21. A method for the prophylactic and/or therapeutic treatment of at least one disorder induced, caused or mediated by Helicobacter pylori infection comprising: administrating to a subject in need thereof a therapeutically effective amount of:
(a) glycine or a pharmaceutically acceptable salt thereof; and
(b) an effective amount of at least one additional Helicobacter pylori therapeutic agent.

22. The method according to claim 21, wherein (a) and (b) are concurrently administered.

23. The method according to claim 21, wherein (a) and (b) are sequentially administered.

24. The method according to claim 21, wherein said Helicobacter pylori is clarithromycin-resistant Helicobacter pylori.

25. The method according to claim 21, wherein said additional Helicobacter pylori therapeutic agent is amoxicillin.

26. The method according to claim 25 wherein said therapeutically effective amount ranges from 500 mg to 30 g of glycine or a pharmaceutically acceptable salt thereof and from 25 mg to 750 mg of amoxicillin administered one to three times per day.

27. The method according to claim 26, wherein said additional Helicobacter pylori therapeutic agent is clarithromycin.

28. The method according to claim 21, wherein said additional Helicobacter pylori therapeutic agent is clarithromycin.

29. The method according to claim 21, further comprising administering:
(c) a therapeutically effective amount of a proton pump inhibitor.

30. The method according to claim 29, wherein said proton pump inhibitor is at least one member selected from the group consisting of omeprazole, esomeprazole, lansoprazole, leminoprazole, rabeprazole, and nepaprazole.

31. The method according to claim 21, further comprising administring:
(d) a therapeutically effective amount of a histamine-H2 receptor blocking compound.

32. The method according to claim 31, wherein said histamine-H2 receptor blocking compound is a member selected from the group consisting of ranitidine, cimetidine, and famotidine.

33. The method according to claim 21, wherein said administering comprises administering
(a) a therapeutically effective amount of glycine or a pharmaceutically acceptable salt thereof;
(b) a therapeutically effective amount of amoxicillin and a therapeutically effective amount of clarithromycin; and
(c) a therapeutically effective amount of a proton pump inhibitor.

34. The method according to claim 21, wherein said administering comprises administering
(a) a therapeutically effective amount of glycine or a pharmaceutically acceptable salt thereof;
(b) a therapeutically effective amount of amoxicillin and a therapeutically effective amount of clarithromycin; and
(d) a therapeutically effective amount of a histamine-H2 receptor blocking compound.


SEQUENCE LISTING (2 sequences)

SEQ ID NO: 1 - DNA, 20 bp, Artificial Sequence
  Synthetic DNA
ccacagcgatgtggtctcag

SEQ ID NO: 2 - DNA, 20 bp, Artificial Sequence
  Synthetic DNA
ctccataagagccaaagccc